(19) United States
(12) Patent Application Publication
(10) Pub. No.: US 2011/0223131 A1
(43) Pub. Date: Sep. 15, 2011

(54) ANTIVIRAL COMPOUNDS

(75) Inventors: Haolun JIN, Foster City, CA (US); Choung U. Kim, San Carlos, CA (US); Barton W. Phillips, San Mateo, CA (US)

(73) Assignee: Gilead Sciences, Inc., Foster City, CA (US)

(21) Appl. No.: 13/032,581

(22) Filed: Feb. 22, 2011

Related U.S. Application Data
(60) Provisional application No. 61/307,707, filed on Feb. 24, 2010.

(51) Int. Cl. A61K 38/21; C07D 498/14; A61K 31/5383; A61K 31/7056; A61P 31/18

(52) U.S. Cl. 424/85.4; 544/101; 514/230.2; 514/43

(57) ABSTRACT

The invention is related to anti-viral compounds, compositions containing such compounds, and therapeutic methods that include the administration of such compounds, as well as to processes and intermediates useful for preparing such compounds.

### FIELD OF THE INVENTION

The invention relates generally to compounds with HIV inhibitory activity.

### BACKGROUND OF THE INVENTION

Human immunodeficiency virus (HIV) is a retrovirus that can lead to acquired immunodeficiency syndrome (AIDS), a condition in humans in which the immune system is weakened, leading to life-threatening opportunistic infections. Although drugs having anti-HIV activity are in wide use and have shown effectiveness, toxicity and other side effects have limited their usefulness. Inhibitors of HIV are useful to limit the establishment and progression of infection by HIV as well as in diagnostic assays for HIV.

There is a need for new HIV therapeutic agents.

### SUMMARY OF THE INVENTION

One aspect of the present invention includes a compound of Formula (I):

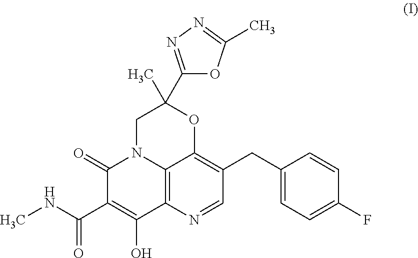

or a pharmaceutically acceptable salt thereof. In one embodiment, a preferred stereochemical configuration provides a compound of Formula (Ia):

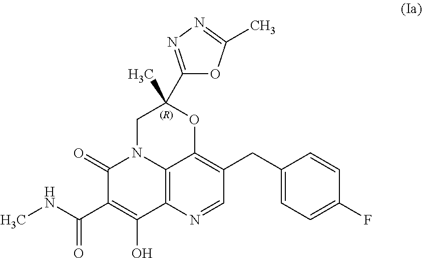

or a pharmaceutically acceptable salt thereof.

Another aspect of the present invention includes a pharmaceutical composition comprising the compound of the present invention and one or more pharmaceutically acceptable excipients. In one embodiment, the pharmaceutical composition further includes one or more additional therapeutic agents. In one embodiment, the additional therapeutic agent is selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, interferons, ribavirin analogs, NS5a inhibitors, NS5b inhibitors, NS3 protease inhibitors, alpha-glucosidase 1 inhibitors, hepatoprotectants, non-nucleoside inhibitors of HIV, and pharmacokinetic enhancers. Furthermore, as will be appreciated, the scope of this aspect of the invention includes therapies yet to be approved, including but not limited to HIV capsid inhibitors and inhibitors of the action of host protein LEDGF.

Another aspect of the present invention includes a method for the treatment or prophylaxis of disorders associated with HIV, comprising administering a therapeutically effective amount of a compound of the present invention. Specifically, an aspect includes delaying the onset or progression of HIV infection. Alternatively, an aspect includes treating HIV infection in a human by administering a therapeutically effective dose. Another aspect includes a compound of the present invention for use in medical therapy. Another aspect includes use of a compound of the present invention for preparing a medicament for treating or preventing HIV, or an HIV-associated disorder. Another aspect includes a compound of the present invention for use in treating or preventing HIV, or an HIV-associated disorder.

Another aspect of the present invention includes administering the compounds or compositions, including combinations, in a once-daily administration scheme. Alternatively, another aspect includes administering the compounds or compositions, including combinations, in a twice-daily administration scheme. Further, another aspect includes a dose range for the compounds in either such administration scheme. In one embodiment, the dose ranges from about 1 to about 6 mg/kg body weight per day for monotherapy and about 1 to about 20 mg/kg body weight per day for combination therapy.

While not wishing to be bound by theory, applicants currently believe that the compounds of formula (I) and (Ia) inhibit the activity of an integrase protein which is encoded by the HIV genome and which is required for the integration of the HIV genome into the genome of a target cell, such as a macrophage or CD4⁺ T cell. Thus, the compounds of the present invention are useful, for example, for inhibiting HIV infection of susceptible human cells.

The present invention also provides a pharmaceutical composition comprising a compound of formula (I) or (Ia), or a pharmaceutically acceptable salt or prodrug thereof, and at least one pharmaceutically acceptable carrier.

The present invention also provides for a method of treating disorders associated with HIV, said method comprising administering to an individual a pharmaceutical composition which comprises a therapeutically effective amount of a compound of formula (I) or (Ia), or a pharmaceutically acceptable salt or prodrug thereof.

The present invention also provides a method of inhibiting HIV, comprising administering to a human afflicted with a condition associated with HIV activity, a therapeutically effective amount of a compound of formula (I) or (Ia), or a pharmaceutically acceptable salt or prodrug thereof, effective to inhibit HIV.

The present invention also provides a compound of formula (I) and (Ia), or a pharmaceutically acceptable salt or prodrug thereof, for use in medical therapy (preferably for use in inhibiting HIV or treating a condition associated with HIV activity), as well as the use of a compound of formula I or Ia, or a pharmaceutically acceptable salt or prodrug thereof, for the manufacture of a medicament useful for inhibiting HIV or the treatment of a condition associated with HIV activity in a mammal, more specifically, a human.

The present invention also provides synthetic processes and novel intermediates disclosed herein which are useful for preparing compounds of the invention. Some of the compounds of the invention are useful to prepare other compounds of the invention.

In another aspect the invention provides a method of inhibiting HIV activity in a sample comprising treating the sample with a compound of formula I, or a pharmaceutically acceptable salt or prodrug thereof.

In one embodiment the invention provides a compound having improved inhibitory or pharmacokinetic properties, including enhanced activity against development of viral resistance, improved oral bioavailability, greater potency, or extended effective half-life in vivo.

The commercial development of a drug candidate involves many steps, including the research and development regarding drug substances that exhibit suitable purity, chemical stability, pharmaceutical properties, and characteristics that facilitate convenient handling and processing. Thus, while potency is a primary goal, additional considerations must be had to any particular drug candidate. One such consideration is solubility. If a drug candidate lacks adequate solubility characteristics, there is likely an associated lack in adequate bioavailability, including but not limited to the relationship between aqueous solubility and oral bioavailability. As demonstrated in more detail below, Compound (Ia) provides a substantial improvement in oral bioavailability as compared to Comparator Compound.

As detailed in the Examples below, the compound of the present invention was compared to a compound of similar activity as was reported in WO 2010/011959. As will be noted, the compounds are both potent anti-virals, however, Compound (Ia) has a significantly lower melting point than that of the Comparator Compound. Moreover, Compound (Ia) exhibits improved solubility over Comparator Compound. Based upon these improved characteristics, the compounds were compared for oral bioavailability. The oral bioavailability for the Comparator Compound was 0.2%±0.2%, whereas the oral bioavailability for Compound (Ia) was 45%±9%. Thus, although each compound demonstrates a preferred level of potency, the lower melting point and increased solubility of Compound (Ia) further provides improved oral bioavailability. The compound of the present invention, therefore, presents an improved profile in terms of the commercial development of a drug candidate.

Combinations of the various aspects, embodiments, and preferences form another aspect of the present invention.

### BRIEF DESCRIPTION OF THE FIGURES

FIG. 1 is a differential scanning calorimetry thermogram for Compound (Ia).

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention as defined by the embodiments.

### DEFINITIONS AND ABBREVIATIONS

### Abbreviations and Acronyms

A list of abbreviations commonly used in the field of organic chemistry appears in The ACS Style Guide (third edition) and in the Guidelines for Authors for the Journal of Organic Chemistry. The chemical elements are identified herein in accordance with the Periodic Table of the Elements, CAS version, Handbook of Chemistry and Physics, 67th Ed., 1986-87. More specifically, and by way of non-limiting example, when the following abbreviations are used herein, they have the following meanings:
¹H-NMR proton nuclear magnetic resonance spectroscopy
³¹P-NMR phosphorus-31 nuclear magnetic resonance spectroscopy
¹⁹F-NMR fluorine-19 nuclear magnetic resonance spectroscopy
AcOH acetic acid
Ac₂O acetic anhydride
abs absolute
aq aqueous
ap approximate
atm atmosphere
br broad
Bu butyl
ACN acetonitrile
Ac₂O acetic anhydride
AcOH acetic acid
Celite® brand of diatomaceous earth from Celite Corp.
CD₃CN acetonitrile-d₃
CD₃OD methanol-d₄
d doublet
DOE dichloroethane
DCM dichloromethane
dd double doublet
DIBAL diisobutylaluminum hydride
DIEA diisopropylethyl amine
DMF N,N-dimethylformamide
DMSO dimethylsulfoxide
DMSO-d₆ dimethyldsulfoxide-d₆
DPPA diphenylphosphoryl azide
equiv equivalent(s)
Et₃N triethylamine
Et₂O diethyl ether
EtOAc ethyl acetate
EtOH ethanol
FBS fetal bovine serum
g gram(s)
h hour(s)
HCl hydrogen chloride
HPLC high performance liquid chromatography
Hz hertz
ISCO® Brand of Medium Pressure Chromatography from ISCO inc.
IPA isopropanol
J NMR coupling constant
L liter(s)
LAH lithium aluminium hydride
LCMS liquid chromatography-mass spectrometry
LHMDS lithium hexamethyldisilazide
L-Selectride lithium tri-sec-butylborohydride
M molar
Me methyl
MeCN acetonitrile
MeOH methanol
mg milligram(s)
MHz megahertz
min minute(s)
mL milliliter
mmol millimole
MPLC medium pressure liquid chromatography
MS mass spectrometry
Ms methanesulfonyl
N normal
NaHMDS sodium hexamethyldisilazide
NBS N-bromosuccinimide
nM nanomolar
Pd—C palladium on carbon
Pr propyl
py-BOP benzotriazol-1-yl-oxytripyrrolidineophosponium hexafluorophosphate
q quartet
Ra—Ni Raney-Nickel
R_{f} TLC retention factor
Rochelle's potassium sodium tartrate salt
RP Reverse phase
RT retention time
rt room temperature
s singlet
t triplet
t-BuOH tert-butanol
TEA triethylamine
TFA trifluoroacetic acid
THF tetrahydrofuran
TLC thin layer chromatography
Ts p-toluenesulfonyl
v/v volume-to-volume proportion
v/v/v volume-to-volume-to-volume proportion
μL microliter
μm micrometer

### Definitions

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. The invention includes all stereoisomers of the compounds described herein.

### Compounds of the Invention

In one aspect the present invention provides compounds of Formula I, or a pharmaceutically acceptable prodrug or salt thereof:

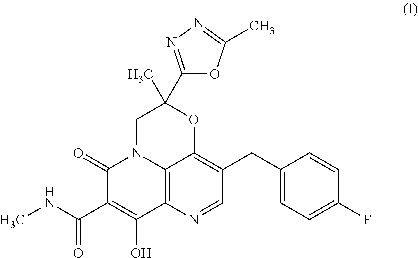

In one embodiment, the present invention provides a preferred stereochemical configuration, namely a compound of Formula (Ia):

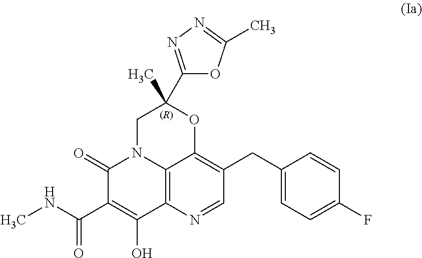

or a pharmaceutically acceptable salt thereof.

### Prodrugs

The present invention includes prodrugs of the compounds of Formula I. The term “prodrug” as used herein refers to any compound that when administered to a biological system generates the drug substance, i.e. active ingredient, as a result of spontaneous chemical reaction(s), enzyme catalyzed chemical reaction(s), photolysis, and/or metabolic chemical reaction(s). A prodrug is thus a covalently modified analog or latent form of a therapeutically-active compound.

“Prodrug moiety” refers to a labile functional group which separates from the active inhibitory compound during metabolism, either systemically or inside a cell, by hydrolysis, enzymatic cleavage, or by some other process (Bundgaard, Hans, “Design and Application of Prodrugs” in A Textbook of Drug Design and Development (1991), P. Krogsgaard-Larsen and H. Bundgaard, Eds. Harwood Academic Publishers, pp. 113-191). Enzymes which are capable of an enzymatic activation mechanism with the phosphonate prodrug compounds of the invention include, but are not limited to, amidases, esterases, microbial enzymes, phospholipases, cholinesterases, and phosphatases. Prodrug moieties can serve to enhance solubility, absorption and lipophilicity to optimize drug delivery, bioavailability and efficacy. A prodrug moiety may include an active metabolite or drug itself.

Exemplary prodrug moieties include the hydrolytically sensitive or labile acyloxymethyl esters —CH₂OC(═O)R⁹ and acyloxymethyl carbonates —CH₂OC(═O)OR⁹ where R⁹ is C₁-C₆ alkyl, C₁-C₆ substituted alkyl, C₆-C₂₀ aryl or C₆-C₂₀ substituted aryl. The acyloxyalkyl ester was first used as a prodrug strategy for carboxylic acids and then applied to phosphates and phosphonates by Farquhar et al. (1983) J. Pharm. Sci. 72: 324; also U.S. Pat. Nos. 4,816,570, 4,968,788, 5,663,159 and 5,792,756. Subsequently, the acyloxyalkyl ester was used to deliver phosphonic acids across cell membranes and to enhance oral bioavailability. A close variant of the acyloxyalkyl ester, the alkoxycarbonyloxyalkyl ester (carbonate), may also enhance oral bioavailability as a prodrug moiety in the compounds of the combinations of the invention. An exemplary acyloxymethyl ester is pivaloyloxymethoxy, (POM) —CH₂OC(═O)C(CH₃)₃. An exemplary acyloxymethyl carbonate prodrug moiety is pivaloyloxymethylcarbonate (POC) —CH₂OC(═O)OC(CH₃)₃.

Aryl esters of phosphorus groups, especially phenyl esters, are reported to enhance oral bioavailability (De Lombaert et al. (1994) J. Med. Chem. 37: 498). Phenyl esters containing a carboxylic ester ortho to a phosphate have also been described (Khamnei and Torrence, (1996) J. Med. Chem. 39:4109-4115). Benzyl esters are reported to generate parent phosphonic acids. In some cases, substituents at the ortho-or para-position may accelerate the hydrolysis. Benzyl analogs with an acylated phenol or an alkylated phenol may generate the phenolic compound through the action of enzymes, e.g., esterases, oxidases, etc., which in turn undergoes cleavage at the benzylic C—O bond to generate phosphoric acid and a quinone methide intermediate. Examples of this class of prodrugs are described by Mitchell et al. (1992) J. Chem. Soc. Perkin Trans. II 2345; Glazier WO 91/19721. Still other benzylic prodrugs have been described containing a carboxylic ester-containing group attached to the benzylic methylene (Glazier WO 91/19721). Thio-containing prodrugs are reported to be useful for the intracellular delivery of phosphonate drugs. These proesters contain an ethylthio group in which the thiol group is either esterified with an acyl group or combined with another thiol group to form a disulfide. Deesterification or reduction of the disulfide generates the free thio intermediate which subsequently breaks down to the phosphoric acid and episulfide (Puech et al. (1993) Antiviral Res., 22: 155-174; Benzaria et al. (1996) J. Med. Chem. 39: 4958).

### Protecting Groups

In the context of the present invention, protecting groups include prodrug moieties and chemical protecting groups.

“Protecting group” refers to a moiety of a compound that masks or alters the properties of a functional group or the properties of the compound as a whole. Chemical protecting groups and strategies for protection/deprotection are well known in the art. See e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. Protection of functional groups of a compound alters other physical properties besides the reactivity of the protected functional group, such as the polarity, lipophilicity (hydrophobicity), and other properties which can be measured by common analytical tools. Chemically protected intermediates may themselves be biologically active or inactive.

Protected compounds may also exhibit altered, and in some cases, optimized properties in vitro and in vivo, such as passage through cellular membranes and resistance to enzymatic degradation or sequestration. In this role, protected compounds with intended therapeutic effects may be referred to as prodrugs. Another function of a protecting group is to convert the parental drug into a prodrug, whereby the parental drug is released upon conversion of the prodrug in vivo. Because active prodrugs may be absorbed more effectively than the parental drug, prodrugs may possess greater potency in vivo than the parental drug. Protecting groups are removed either in vitro, in the instance of chemical intermediates, or in vivo, in the case of prodrugs. With chemical intermediates, it is not particularly important that the resulting products after deprotection, e.g., alcohols, be physiologically acceptable, although in general it is more desirable if the products are pharmacologically innocuous.

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene (John Wiley & Sons, Inc., New York, 1991, ISBN 0-471-62301-6) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene.

### Stereoisomers

The compounds of the invention may have chiral centers, e.g., chiral carbon or phosphorus atoms. The compounds of the invention thus include racemic mixtures of all stereoisomers, including enantiomers, diastereomers, and atropisomers. In addition, the compounds of the invention include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the invention. The racemic mixtures are separated into their individual, substantially optically pure isomers through well-known techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

The compounds of the invention can also exist as tautomeric isomers in certain cases. Although only one delocalized resonance structure may be depicted, all such forms are contemplated within the scope of the invention.

### Salts and Hydrates

Examples of physiologically acceptable salts of the compounds of the invention include salts derived from an appropriate base, such as an alkali metal (for example, sodium), an alkaline earth (for example, magnesium), ammonium and NX₄⁺ (wherein X is C₁-C₄ alkyl). Physiologically acceptable salts of an hydrogen atom or an amino group include salts of organic carboxylic acids such as acetic, benzoic, citric, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; and inorganic acids, such as hydrochloric, sulfuric, phosphoric and sulfamic acids. Physiologically acceptable salts of a compound of a hydroxy group include the anion of said compound in combination with a suitable cation such as Na⁺ and NX₄⁺ (wherein X is independently selected from H or a C₁-C₄ alkyl group).

For therapeutic use, salts of active ingredients of the compounds of the invention will typically be physiologically acceptable, i.e. they will be salts derived from a physiologically acceptable acid or base. However, salts of acids or bases which are not physiologically acceptable may also find use, for example, in the preparation or purification of a physiologically acceptable compound. All salts, whether or not derived from a physiologically acceptable acid or base, are within the scope of the present invention.

Metal salts typically are prepared by reacting the metal hydroxide with a compound of this invention. Examples of metal salts which are prepared in this way are salts containing Li⁺, Na⁺, and K⁺. A less soluble metal salt can be precipitated from the solution of a more soluble salt by addition of the suitable metal compound.

In addition, salts may be formed from acid addition of certain organic and inorganic acids, e.g., HCl, HBr, H₂SO₄, H₃PO₄ or organic sulfonic acids, to basic centers, typically amines, or to acidic groups. Finally, it is to be understood that the compositions herein comprise compounds of the invention in their un-ionized, as well as zwitterionic form, and combinations with stoichiometric amounts of water as in hydrates.

### Methods of Inhibition of HIV

Another aspect of the invention relates to methods of inhibiting the activity of HIV comprising the step of treating a sample suspected of containing HIV with a compound or composition of the invention.

Prevention of HIV-related disorders may be manifested by delaying or preventing the progression of the disorder, as well as the onset of the symptoms associated with the disorder. Treatment of the disorder may be manifested by a decrease or elimination of symptoms, inhibition or reversal of the progression of the disorder, as well as any other contribution to the well being of the patient.

Compounds of the invention may act as inhibitors of HIV, as intermediates for such inhibitors or have other utilities as described below. The inhibitors will generally bind to one or more locations on the HIV integrase protein. Compounds may bind with varying degrees of reversibility. Those compounds binding substantially irreversibly are useful as probes for identifying the presence of HIV. Accordingly, the invention relates to methods of detecting HIV in a sample suspected of containing HIV comprising the steps of: treating a sample suspected of containing HIV with a composition comprising a compound of the invention bound to a label; and observing the effect of the sample on the activity of the label. Suitable labels are well known in the diagnostics field and include stable free radicals, fluorophores, radioisotopes, enzymes, chemiluminescent groups and chromogens. The compounds herein are labeled in conventional fashion using functional groups such as hydroxyl or amino. In one embodiment the invention provides a compound of formula (I) that comprises or that is bound or linked to one or more detectable labels. Within the context of the invention samples suspected of containing HIV include natural or man-made materials such as living organisms; tissue or cell cultures; biological samples such as biological material samples (blood, serum, urine, cerebrospinal fluid, tears, sputum, saliva, tissue samples, and the like); laboratory samples; food, water, or air samples; bioproduct samples such as extracts of cells, particularly recombinant cells synthesizing a desired glycoprotein; and the like. Typically the sample will be suspected of containing HIV. Samples can be contained in any medium including water and organic solvent/water mixtures. Samples include living organisms such as humans, and man made materials such as cell cultures.

The treating step of the invention comprises adding the compound of the invention to the sample or it comprises adding a precursor of the composition to the sample. The addition step comprises any method of administration as described above.

If desired, the activity of HIV after application of the compound can be observed by any method including direct and indirect methods of detecting HIV activity. Quantitative, qualitative, and semiquantitative methods of determining HIV activity are all contemplated. Typically one of the screening methods described above are applied, however, any other method such as observation of the physiological properties of a living organism are also applicable.

The compounds of this invention are useful in the treatment or prophylaxis of conditions associated with HIV in man.

However, in screening compounds capable of inhibiting HIV it should be kept in mind that the results of enzyme assays may not always correlate with cell culture assays. Thus, a cell based assay should typically be the primary screening tool.

In another aspect, the present invention provides methods of treating AIDS and/or treating disorders associated with AIDS. Non-limiting examples of disorders associated with HIV infection include pneumonia, dementia, various neuropathies, lymphomas and cancer, pain, and opportunistic infections. The methods of this aspect of the invention each include the step of administering to a human being infected with HIV a pharmaceutical composition which includes a therapeutically effective amount of a compound of the present invention. The therapeutically effective amount of the compound of the present invention reduces the rate of replication of HIV, in some instances completely inhibiting the replication of HIV in the infected person. Compounds of the present invention are typically administered in the form of pharmaceutical formulations as described herein in the section entitled “Pharmaceutical Formulations”.

### Screens for HIV Inhibitors

Compounds of the invention are screened for inhibitory activity against HIV by any of the conventional techniques for evaluating enzyme activity. Within the context of the invention, typically compounds are first screened for inhibition of HIV in vitro and compounds showing inhibitory activity are then screened for activity in vivo. Compounds having in vitro Ki (inhibitory constants) of less than about 5×10⁻⁶ M, typically less than about 1×10⁻⁷ M and preferably less than about 5×10⁻⁸ M are preferred for in vivo use.

Representative examples of assays useful for measuring the anti-HIV activity of compounds of the present invention include the assays and methods described in the following publications which are each incorporated herein by reference: Wolfe, et al J. Virol. (1996) 70:1424-1432; Hazuda, et al Nucleic Acids Res. (1994) 22:1121-22; Hazuda, et al J. Virol. (1997) 71:7005-7011; Hazuda, et al Drug Design and Discovery (1997) 15:17-24; and Hazuda, et al Science (2000) 287:646-650.

### Pharmaceutical Formulations

The compounds of this invention are formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986). Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations of the invention comprise at least one active ingredient, as above defined, together with one or more acceptable carriers therefor and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 pg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of conditions associated with HIV activity.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

Effective dose of active ingredient depends at least on the nature of the condition being treated, toxicity, whether the compound is being used prophylactically (lower doses), the method of delivery, and the pharmaceutical formulation, and will be determined by the clinician using conventional dose escalation studies. It can be expected to be from about 0.0001 to about 100 mg/kg body weight per day. Typically, from about 0.01 to about 10 mg/kg body weight per day. More typically, from about 0.01 to about 5 mg/kg body weight per day. More typically, from about 0.05 to about 0.5 mg/kg body weight per day. For example, the daily candidate dose for an adult human of approximately 70 kg body weight will range from 1 mg to 1000 mg, preferably between 5 mg and 500 mg, and may take the form of single or multiple doses.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Combination Therapy

A compound of the invention may be employed in combination with other therapeutic agents for the treatment or prophylaxis of AIDS and/or one or more other diseases present in a human subject suffering from AIDS (e.g., bacterial and/or fungal infections, other viral infections such as hepatitis B or hepatitis C, or cancers such as Kaposi's sarcoma). The additional therapeutic agent(s) may be co-formulated with one or more compounds of the invention (e.g., co-formulated in a tablet).

Examples of such additional therapeutic agents include agents that are effective for the treatment or prophylaxis of viral, parasitic or bacterial infections, or associated conditions, or for treatment of tumors or related conditions, include 3′-azido-3′-deoxythymidine (zidovudine, AZT), 2′-deoxy-3′-thiacytidine (3TC), 2′,3′-dideoxy-2′,3′-didehydroadenosine (D4A), 2′,3′-dideoxy-2′,3′-didehydrothymidine (D4T), carbovir (carbocyclic 2′,3′-dideoxy-2′,3′-didehydroguanosine), 3′-azido-2′,3′-dideoxyuridine, 5-fluorothymidine, (E)-5-(2-bromovinyl)-2′-deoxyuridine (BVDU), 2-chlorodeoxyadenosine, 2-deoxycoformycin, 5-fluorouracil, 5-fluorouridine, 5-fluoro-2′-deoxyuridine, 5-trifluoromethyl-2′-deoxyuridine, 6-azauridine, 5-fluoroorotic acid, methotrexate, triacetyluridine, 1-(2′-deoxy-2′-fluoro-1-β-arabinosyl)-5-iodocytidine (FIAC), tetrahydro-imidazo(4,5,1-jk)-(1,4)-benzodiazepin-2(1H)-thione (TIBO), 2′-nor-cyclicGMP, 6-methoxypurine arabinoside (ara-M), 6-methoxypurine arabinoside 2′-O-valerate; cytosine arabinoside (ara-C), 2′,3′-dideoxynucleosides such as 2′,3′-dideoxycytidine (ddC), 2′,3′-dideoxyadenosine (ddA) and 2′,3′-dideoxyinosine (ddl); acyclic nucleosides such as acyclovir, penciclovir, famciclovir, ganciclovir, HPMPC, PMEA, PMEG, PMPA, PMPDAP, FPMPA, HPMPA, HPMPDAP, (2R,5R)-9→tetrahydro-5-(phosphonomethoxy)-2-furanyladenine, (2R,5R)-1→tetrahydro-5-(phosphonomethoxy)-2-furanylthymine; other antivirals including ribavirin (adenine arabinoside), 2-thio-6-azauridine, tubercidin, aurintricarboxylic acid, 3-deazaneoplanocin, neoplanocin, rimantidine, adamantine, and foscarnet (trisodium phosphonoformate); antibacterial agents including bactericidal fluoroquinolones (ciprofloxacin, pefloxacin and the like); aminoglycoside bactericidal antibiotics (streptomycin, gentamicin, amicacin and the like); β-lactamase inhibitors (cephalosporins, penicillins and the like); other antibacterials including tetracycline, isoniazid, rifampin, cefoperazone, claithromycin and azithromycin, antiparasite or antifungal agents including pentamidine(1,5-bis(4′-aminophenoxy)pentane), 9-deaza-inosine, sulfamethoxazole, sulfadiazine, quinapyramine, quinine, fluconazole, ketoconazole, itraconazole, Amphotericin B, 5-fluorocytosine, clotrimazole, hexadecylphosphocholine and nystatin; renal excretion inhibitors such as probenicid; nucleoside transport inhibitors such as dipyridamole, dilazep and nitrobenzylthioinosine, immunomodulators such as FK506, cyclosporin A, thymosin α-1; cytokines including TNF and TGF-β; interferons including IFN-α, IFN-β, and IFN-γ; interleukins including various interleukins, macrophage/granulocyte colony stimulating factors including GM-CSF, G-CSF, M-CSF, cytokine antagonists including anti-TNF antibodies, anti-interleukin antibodies, soluble interleukin receptors, protein kinase C inhibitors and the like.

Examples of suitable active therapeutic agents or ingredients which can be combined with one or more compounds of the invention, and which have activity against HIV, include 1) HIV protease inhibitors, e.g., amprenavir, atazanavir, fosamprenavir, indinavir, lopinavir, ritonavir, lopinavir+ritonavir, nelfinavir, saquinavir, tipranavir, brecanavir, darunavir, TMC-126, TMC-114, mozenavir (DMP-450), JE-2147 (AG1776), AG1859, DG35, L-756423, RO0334649, KNI-272, DPC-681, DPC-684, and GW640385X, DG17, PPL-100, 2) a HIV non-nucleoside inhibitor of reverse transcriptase, e.g., capravirine, emivirine, delaviridine, efavirenz, nevirapine, (+) calanolide A, etravirine, GW5634, DPC-083, DPC-961, DPC-963, MIV-150, and TMC-120, TMC-278 (rilpivirine), efavirenz, BILR 355 BS, VRX 840773, UK-453,061, RDEA806, 3) a HIV nucleoside inhibitor of reverse transcriptase, e.g., zidovudine, emtricitabine, didanosine, stavudine, zalcitabine, lamivudine, abacavir, amdoxovir, elvucitabine, alovudine, MIV-210, racivir (-FTC), D-d4FC, emtricitabine, phosphazide, fozivudine tidoxil, fosalvudine tidoxil, apricitibine (AVX754), amdoxovir, KP-1461, abacavir+lamivudine, abacavir+lamivudine+zidovudine, zidovudine+lamivudine, 4) a HIV nucleotide inhibitor of reverse transcriptase, e.g., tenofovir, tenofovir disoproxil fumarate+emtricitabine, tenofovir disoproxil fumarate+emtricitabine+efavirenz, and adefovir, 5) a HIV integrase inhibitor, e.g., curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, S-1360, zintevir (AR-177), L-870812, and L-870810, MK-0518 (raltegravir), BMS-707035, MK-2048, BA-011, BMS-538158, GSK364735C, 6) a gp41 inhibitor, e.g., enfuvirtide, sifuvirtide, FB006M, TRI-1144, SPC3, DES6, Locus gp41, CovX, and REP 9, 7) a CXCR4 inhibitor, e.g., AMD-070, 8) an entry inhibitor, e.g., SP01A, TNX-355, 9) a gp120 inhibitor, e.g., BMS-488043 and BlockAide/CR, 10) a G6PD and NADH-oxidase inhibitor, e.g., immunitin, 10) a CCR5 inhibitor, e.g., aplaviroc, vicriviroc, INCB9471, PRO-140, INCB15050, PF-232798, CCR5mAb004, and maraviroc, 11) an interferon, e.g., pegylated rIFN-alpha 2b, pegylated rIFN-alpha 2a, rIFN-alpha 2b, IFN alpha-2b XL, rIFN-alpha 2a, consensus IFN alpha, infergen, rebif, locteron, AVI-005, PEG-infergen, pegylated IFN-beta, oral interferon alpha, feron, reaferon, intermax alpha, r-IFN-beta, infergen+actimmune, IFN-omega with DUROS, and albuferon, 12) ribavirin analogs, e.g., rebetol, copegus, levovirin, VX-497, and viramidine (taribavirin) 13) NS5a inhibitors, e.g., A-831 and A-689, 14) NS5b polymerase inhibitors, e.g., NM-283, valopicitabine, R1626, PSI-6130 (R1656), HIV-796, BILB 1941, MK-0608, NM-107, R7128, VCH-759, PF-868554, GSK625433, and XTL-2125, 15) NS3 protease inhibitors, e.g., SCH-503034 (SCH-7), VX-950 (Telaprevir), ITMN-191, and BILN-2065, 16) alpha-glucosidase 1 inhibitors, e.g., MX-3253 (celgosivir) and UT-231B, 17) hepatoprotectants, e.g., IDN-6556, ME 3738, MitoQ, and LB-84451, 18) non-nucleoside inhibitors of HIV, e.g., benzimidazole derivatives, benzo-1,2,4-thiadiazine derivatives, and phenylalanine derivatives, 19) other drugs for treating HIV, e.g., zadaxin, nitazoxanide (alinea), BIVN-401 (virostat), DEBIO-025, VGX-410C, EMZ-702, AVI 4065, bavituximab, oglufanide, PYN-17, KPE02003002, actilon (CPG-10101), KRN-7000, civacir, GI-5005, ANA-975 (isatoribine), XTL-6865, ANA 971, NOV-205, tarvacin, EHC-18, and NIM811, 19) pharmacokinetic enhancers (molecules that enhance the bioavailability of another drug by inhibiting the metabolism of the other drug which is typically coadministered with the pharmacokinetic enhancer), e.g., BAS-100, PF-4194477, TMC-41629, roxythromycin and SPI452, 20) RNAse H inhibitors, e.g., ODN-93 and ODN-112, 21) other anti-HIV agents, e.g., VGV-1, PA-457 (bevirimat), ampligen, HRG214, cytolin, polymun, VGX-410, KD247, AMZ 0026, CYT 99007, A-221 HIV, BAY 50-4798, MDX010 (iplimumab), PBS119, ALG889, and PA-1050040.

Again by way of example, the following list discloses exemplary HIV antivirals, with their corresponding U.S. Patent Numbers for synthetic reference, which can be combined with one or more of the compounds of the present invention.

### Exemplary HIV Antivirals and Patent Numbers

Ziagen (Abacavir sulfate, U.S. Pat. No. 5,034,394)
Epzicom (Abacavir sulfate/lamivudine, U.S. Pat. No. 5,034,394)
Hepsera (Adefovir dipivoxil, U.S. Pat. No. 4,724,233)
Agenerase (Amprenavir, U.S. Pat. No. 5,646,180)
Reyataz (Atazanavir sulfate, U.S. Pat. No. 5,849,911)
Rescriptor (Delavirdine mesilate, U.S. Pat. No. 5,563,142)
Hivid (Dideoxycytidine; Zalcitabine, U.S. Pat. No. 5,028,595)
Videx (Dideoxyinosine; Didanosine, U.S. Pat. No. 4,861,759)
Sustiva (Efavirenz, U.S. Pat. No. 5,519,021)
Emtriva (Emtricitabine, U.S. Pat. No. 6,642,245)
Lexiva (Fosamprenavir calcium, U.S. Pat. No. 6,436,989)
Virudin; Triapten; Foscavir (Foscarnet sodium, U.S. Pat. No. 6,476,009)
Crixivan (Indinavir sulfate, U.S. Pat. No. 5,413,999)
Epivir (Lamivudine, U.S. Pat. No. 5,047,407)
Combivir (Lamivudine/Zidovudine, U.S. Pat. No. 4,724,232)
Aluviran (Lopinavir)
Kaletra (Lopinavir/ritonavir, U.S. Pat. No. 5,541,206)
Viracept (Nelfinavir mesilate, U.S. Pat. No. 5,484,926)
Viramune (Nevirapine, U.S. Pat. No. 5,366,972)
Norvir (Ritonavir, U.S. Pat. No. 5,541,206)
Invirase; Fortovase (Saquinavir mesilate, U.S. Pat. No. 5,196,438)
Zerit (Stavudine, U.S. Pat. No. 4,978,655)
Truvada (Tenofovir disoproxil fumarate/emtricitabine, U.S. Pat. No. 5,210,085)
Aptivus (Tipranavir)
Retrovir (Zidovudine; Azidothymidine, U.S. Pat. No. 4,724,232)

In yet another embodiment, the present invention provides a combination pharmaceutical agent comprising:

a) a first pharmaceutical composition comprising a compound of the present invention, or a pharmaceutically acceptable salt, solvate, or ester thereof; and

b) a second pharmaceutical composition comprising at least one additional therapeutic agent selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, interferons, ribavirin analogs, NS5a inhibitors, NS5b inhibitors, NS3 protease inhibitors, alpha-glucosidase 1 inhibitors, hepatoprotectants, non-nucleoside inhibitors of HIV, pharmacokinetic enhancers, and other drugs for treating HIV. This embodiment, as well, includes combinations thereof.

### Exemplary Methods of Making the Compounds of the Invention

The invention also relates to methods of making the compositions of the invention. The compositions are prepared by any of the applicable techniques of organic synthesis. Many such techniques are well known in the art. However, many of the known techniques are elaborated in Compendium of Organic Synthetic Methods (John Wiley & Sons, New York), Vol. 1, Ian T. Harrison and Shuyen Harrison, 1971; Vol. 2, Ian T. Harrison and Shuyen Harrison, 1974; Vol. 3, Louis S. Hegedus and Leroy Wade, 1977; Vol. 4, Leroy G. Wade, Jr., 1980; Vol. 5, Leroy G. Wade, Jr., 1984; and Vol. 6, Michael B. Smith; as well as March, J., Advanced Organic Chemistry, Third Edition, (John Wiley & Sons, New York, 1985), Comprehensive Organic Synthesis. Selectivity, Strategy & Efficiency in Modern Organic Chemistry. In 9 Volumes, Barry M. Trost, Editor-in-Chief (Pergamon Press, New York, 1993 printing).

A number of exemplary methods for the preparation of the compositions of the invention are provided below. These methods are intended to illustrate the nature of such preparations and are not intended to limit the scope of applicable methods.

Generally, the reaction conditions such as temperature, reaction time, solvents, work-up procedures, and the like, will be those common in the art for the particular reaction to be performed. The cited reference material, together with material cited therein, contains detailed descriptions of such conditions. Typically the temperatures will be −100° C. to 200° C., solvents will be aprotic or protic, and reaction times will be 10 seconds to 10 days. Work-up typically consists of quenching any unreacted reagents followed by partition between a water/organic layer system (extraction) and separating the layer containing the product.

Oxidation and reduction reactions are typically carried out at temperatures near room temperature (about 20° C.), although for metal hydride reductions frequently the temperature is reduced to 0° C. to −100° C., solvents are typically aprotic for reductions and may be either protic or aprotic for oxidations. Reaction times are adjusted to achieve desired conversions.

Condensation reactions are typically carried out at temperatures near room temperature, although for non-equilibrating, kinetically controlled condensations reduced temperatures (0° C. to −100° C.) are also common. Solvents can be either protic (common in equilibrating reactions) or aprotic (common in kinetically controlled reactions).

Standard synthetic techniques such as azeotropic removal of reaction by-products and use of anhydrous reaction conditions (e.g., inert gas environments) are common in the art and will be applied when applicable.

The terms “treated”, “treating”, “treatment”, and the like, when used in connection with a chemical synthetic operation, mean contacting, mixing, reacting, allowing to react, bringing into contact, and other terms common in the art for indicating that one or more chemical entities is treated in such a manner as to convert it to one or more other chemical entities. This means that “treating compound one with compound two” is synonymous with “allowing compound one to react with compound two”, “contacting compound one with compound two”, “reacting compound one with compound two”, and other expressions common in the art of organic synthesis for reasonably indicating that compound one was “treated”, “reacted”, “allowed to react”, etc., with compound two. For example, treating indicates the reasonable and usual manner in which organic chemicals are allowed to react. Normal concentrations (0.01M to 10M, typically 0.1M to 1M), temperatures (−100° C. to 250° C., typically −78° C. to 150° C., more typically −78° C. to 100° C., still more typically 0° C. to 100° C.), reaction vessels (typically glass, plastic, metal), solvents, pressures, atmospheres (typically air for oxygen and water insensitive reactions or nitrogen or argon for oxygen or water sensitive), etc., are intended unless otherwise indicated. The knowledge of similar reactions known in the art of organic synthesis is used in selecting the conditions and apparatus for “treating” in a given process. In particular, one of ordinary skill in the art of organic synthesis selects conditions and apparatus reasonably expected to successfully carry out the chemical reactions of the described processes based on the knowledge in the art.

Modifications of each of the exemplary schemes and examples (hereinafter collectively referred to as “exemplary schemes”) leads to various analogs of the exemplified materials. The above-cited citations describing suitable methods of organic synthesis are applicable to such modifications.

In each of the exemplary schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium, and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point, and molecular weight in distillation and sublimation, presence or absence of polar functional groups in chromatography, stability of materials in acidic and basic media in multiphase extraction, and the like. One skilled in the art will apply techniques most likely to achieve the desired separation.

A single stereoisomer, e.g., an enantiomer, substantially free of its stereoisomer may be obtained by resolution of the racemic mixture using a method such as formation of diastereomers using optically active resolving agents (Stereochemistry of Carbon Compounds, (1962) by E. L. Eliel, McGraw Hill; Lochmuller, C. H., (1975) J. Chromatogr., 113) 283-302). Racemic mixtures of chiral compounds of the invention can be separated and isolated by any suitable method, including: formation of ionic, diastereomeric salts with chiral compounds and separation by fractional crystallization or other methods; formation of diastereomeric compounds with chiral derivatizing reagents, separation of the diastereomers, and conversion to the pure stereoisomers; or separation of the substantially pure or enriched stereoisomers directly under chiral conditions.

Diastereomeric salts can be formed by reaction of enantiomerically pure chiral bases such as brucine, quinine, ephedrine, strychnine, α-methyl-β-phenylethylamine(amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, the substrate to be resolved is reacted with one enantiomer of a chiral compound to form a diastereomeric pair (Eliel, E. and Wilen, S. (1994) Stereochemistry of Organic Compounds, John Wiley & Sons, Inc., p. 322). Diastereomeric compounds can be formed by reacting asymmetric compounds with enantiomerically pure chiral derivatizing reagents, such as menthyl derivatives, followed by separation of the diastereomers and hydrolysis to yield the free, enantiomerically enriched xanthene. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric diastereomers. Stable diastereomers of atropisomeric compounds can be separated and isolated by normal- and reverse-phase chromatography following methods for separation of atropisomeric naphthyl-isoquinolines (Hoye, T., WO 96/15111).

Additionally, a racemic mixture of two enantiomers can be separated by chromatography using a chiral stationary phase (Chiral Liquid Chromatography (1989) W. J. Lough, Ed. Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

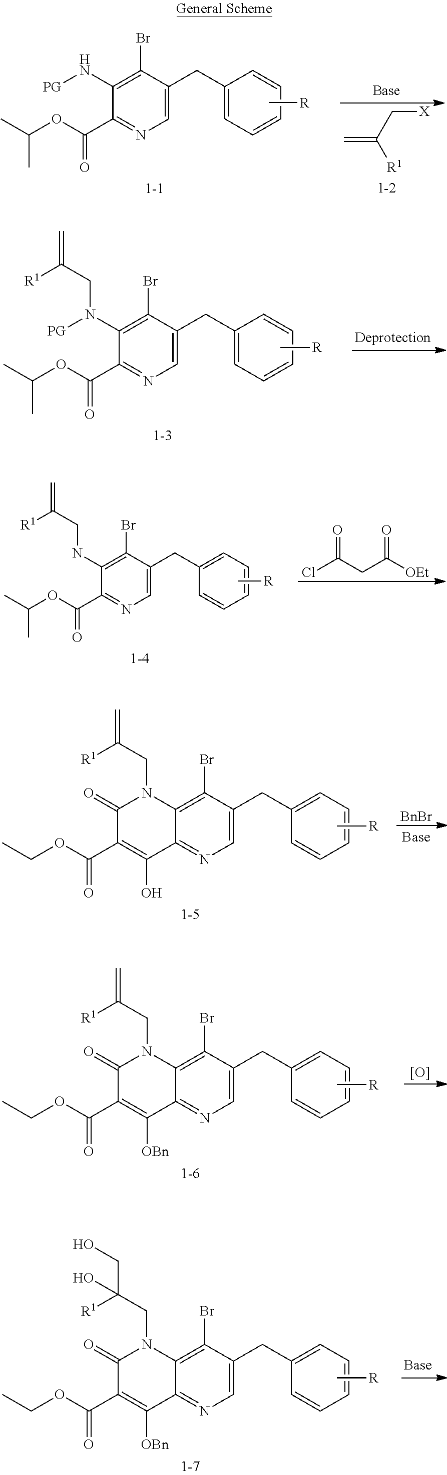

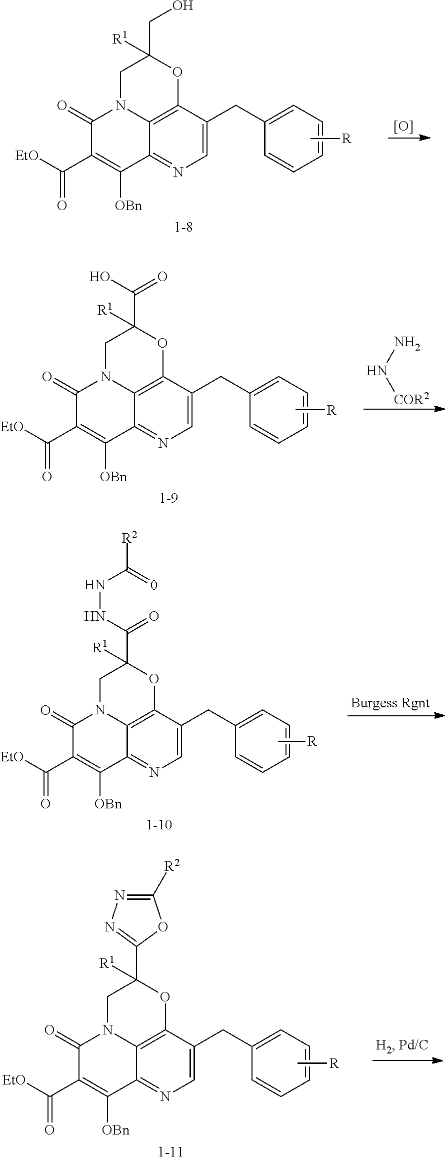

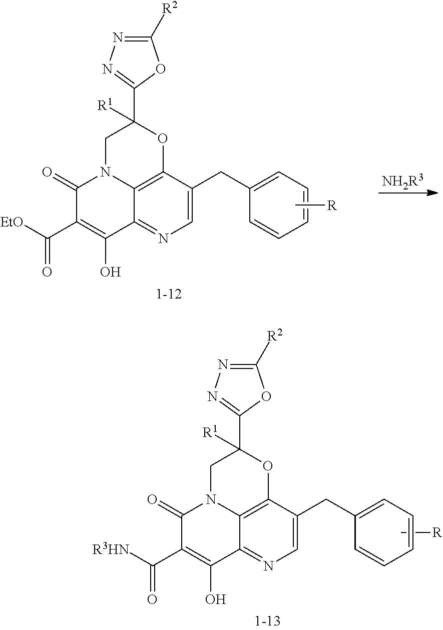

The Compounds of Formula 1-13 can be prepared by the methods described in the General Scheme. Treatment of suitably protected compounds of Formula 1-1, where PG is a carbamate protecting group such as Boc or the like, with an alkyl halide of formula 1-2, where R¹ is alkyl, in the presence of a suitable base provides compounds of Formula 1-3. Removal of the nitrogen protecting group provides compounds of Formula 1-4. Acylation with ethyl malonyl chloride, followed by treatment with a suitable base, such as sodium ethoxide or the like, provides bicyclic heterocycles of Formula 1-5. Subsequent allkylation with a suitable alkyl protecting group, preferably BnBr, in the presence of a base, preferably silver oxide, provides compounds of Formula 1-6. Oxidation of the alkene functionality with a suitable reagent, preferably osmium tetroxide, provides compounds of Formula 1-7. Subsequent treatment with a suitable base, such as DBU or the like, provides tricyclic heterocycles of Formula 1-8. Oxidation of the alcohol functionality with a suitable oxidizing agent, such as TEMPO, provides compounds of Formula 1-9. Treatment with an appropriate acetyl hydrazide in the presence of a suitable coupling reagent such as py-BOP or the like, provides compounds of formula 1-10. Treating compounds of formula 1-10 with a dehydrating agent such as Burgess reagent, provides compounds of formula 1-11. Deprotection of the benzyl ether by hydrogenolysis provides compounds of formula 1-12. Finally, exposure to an appropriate amine in the presence of heat provides compounds of Formula 1-13.

### EXAMPLE 1

### Preparation of Intermediate I-H

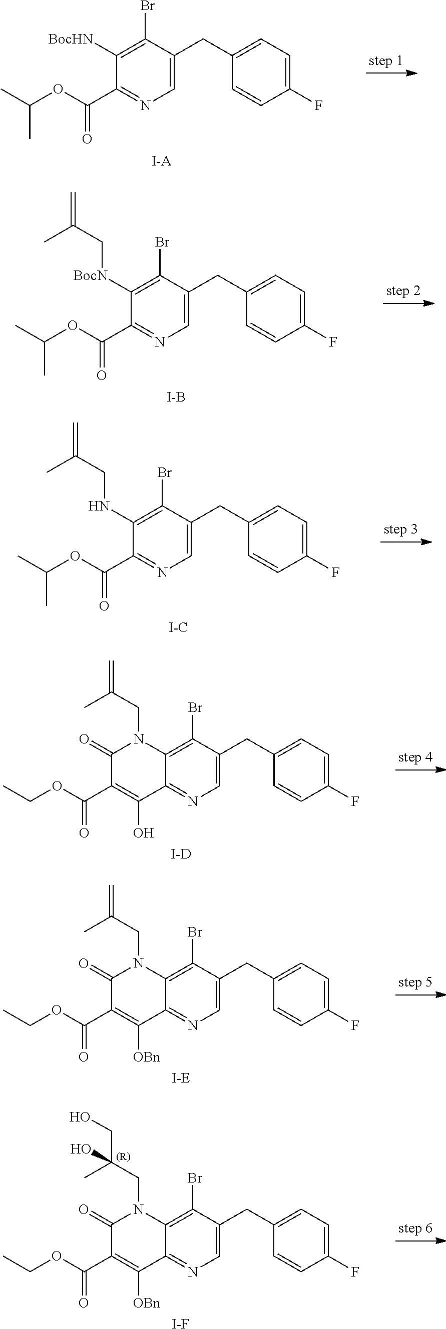

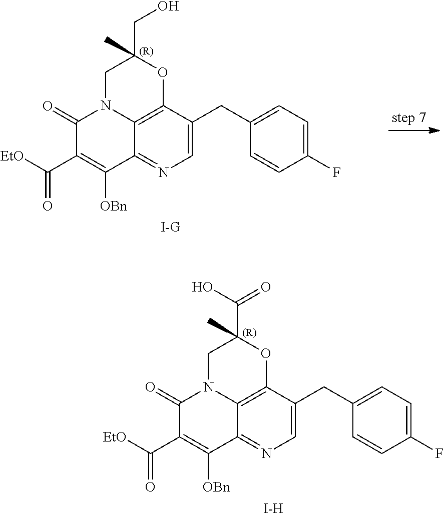

### Steps 1-7

Intermediate I-H was prepared using the same procedures described in example 68, WO 2010/011959, herein incorporated by reference in its entirety. Intermediate I-H (10.79 g, 20.3 mmol) was added portionwise to 100 mL refluxing IPA with stirring. Additional IPA (20 mL) was added until all the solid had completely dissolved. The mixture was then covered with an insulating blanket to allow slow cooling to rt. After 5 min, seed crystals were introduced, and the mixture allowed to stand overnight. The suspension was filtered to provide 5.71 g (53% Yield) as a pale yellow, crystalline solid. ¹H-NMR (DMSO) d 13.62 (bs, 1H), 8.43 (s, 1H), 7.41-7.30 (m, 7H), 7.11-7.05 (m, 2H), 5.60 (app q, J=14 Hz, 2H), 4.88 (d, J=14 Hz, 1H), 4.16 (q, J=7 Hz, 2H), 4.06-4.04 (m, 2H), 3.67 (d, J=14 hz, 1H), 1.76 (s, 3H), 1.14 (t, J=7 Hz, 3H); MS [M+H]=533.1.

### EXAMPLE 2

### Preparation of Compound (Ia)

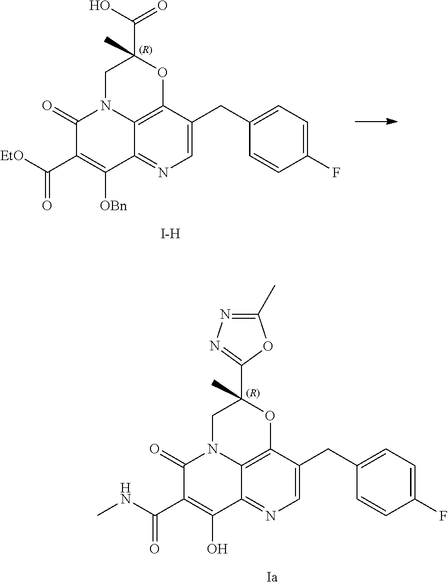

### Step 1

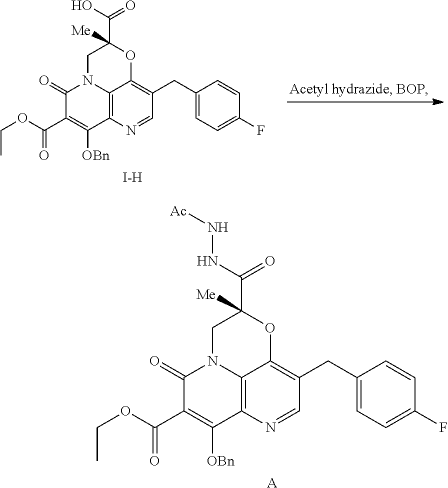

Intermediate I-H (859 mg, 1.61 mmol) was taken up in 12 mL of DMF and treated with py-BOP (1.48 g, 2.85 mmol), N-acetylhydrazide (211 mg, 2.85 mmol), and NMM (678 mg, 6.7 mmol). After stirring for 1 h, the reaction mixture was diluted with 200 mL EtOAc and washed successively with 10% citric acid, 10% sodium citrate, 2.5% LiCl and brine. The organics were dried over sodium sulfate and concentrated in vacuo to provide the desired product A as an orange semi-solid. MS [M+H]+=589.00 LCMS RT=2.42 min.

### Step 2

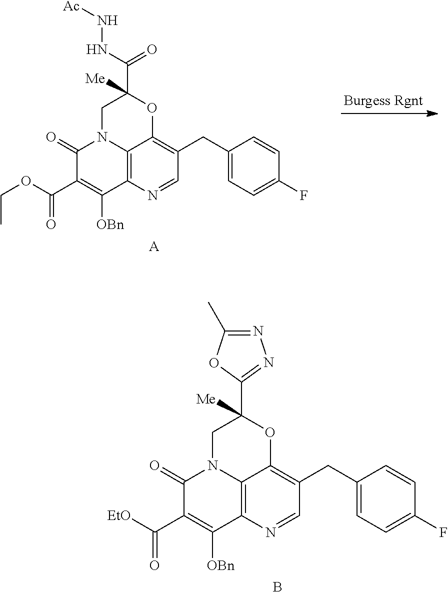

Intermediate A (945 mg, 1.61 mmol) was taken up in 30 mL of dry THF and treated with Burgess Reagent (1.67 g, 7.3 mmol). The reaction was stirred for 3 h at 60° C. The reaction mixture was applied directly to a silica gel loading column (ISCO) and purified by silica gel chromatography (100% DCM to 100% EtOAc), to provide the desired product B (670 mg, 73% Yield, 2 steps) as a white foam. MS [M+H]+=571.00 LCMS RT=2.52 min.

### Step 3

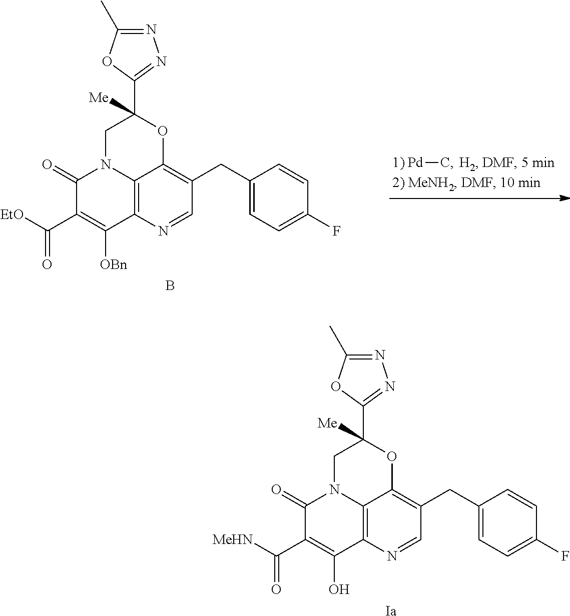

Intermediate B (670 mg, 1.17 mmol) was stirred with 200 mg 10% Pd—C in 5.5 mL of DMF under an atmosphere of H₂ for 5 min. The reaction mixture was filtered thru a 0.45 μM filter to remove the catalyst, and the filtrate was treated with MeNH₂ (10 mL, 20 mmol, 2.0 M) and heated at 125° C. in a μ-wave reactor for 10 min. The reaction mixture was concentrated in vacuo and the residue taken up in EtOAc and washed successively with 10% citric acid, water (3×), 5% LiCl and brine. Concentration of the organics in vacuo and trituration with ether provided the desired product Ia (503 mg, 92% Yield, 2 steps) as an amorphous solid. To obtain crystalline material, the amorphous solid was suspended in 4.5 mL EtOAc and heated to reflux. 1.5 mL hexanes were added dropwise, and the mixture was allowed to cool slowly to rt. After 15 min the solution was treated with 0.1 mg of previously prepared seed crystals. Over the next 2 h, the reaction mixture formed a thick suspension. This mixture was then stirred at rt overnight. Filtration provided the desired product (197 mg, 39% yield) as white crystals (mp. 176° C.). Cooling the filtrate to −10° C. provided a second crop of crystals (131 mg, 26% yield). ¹H-NMR (400 MHz, DMSO-d₆) d 9.99 (bs, 1H), 8.48 (s, 1H), 7.27-7.22 (m, 2H), 7.07 (app t, J=9 Hz, 2H), 5.05 (d, J=14 Hz, 1H), 4.04-3.96 (m, 3H), 2.89 (d, J=5 Hz, 3H), 2.32 (s, 3H), 1.86 (s, 3H); MS [M+H]+=466.0 LCMS RT=2.29 min.

### EXAMPLE 3

### Comparator Compound

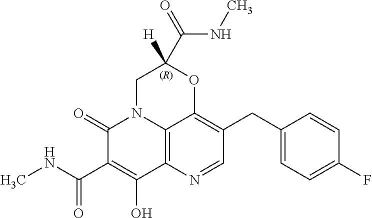

Comparator Compound was made using the procedure set forth in Example 44 (Compound A50) of WO 2010/011959, herein incorporated by reference in its entirety.

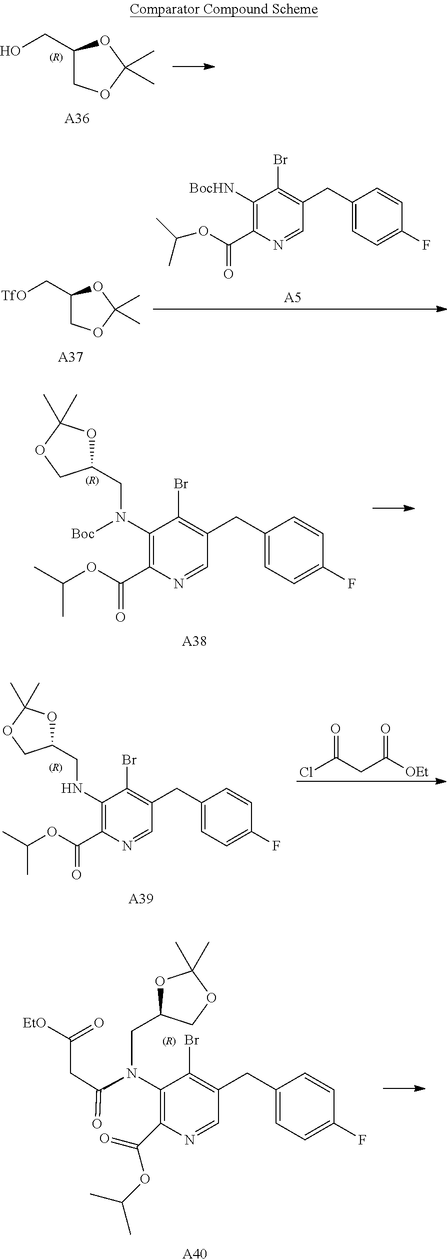

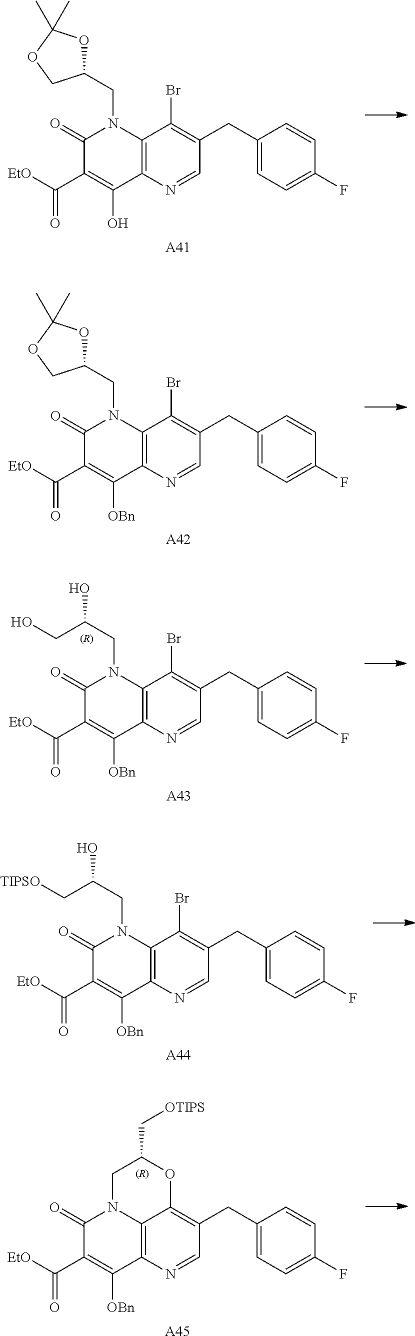

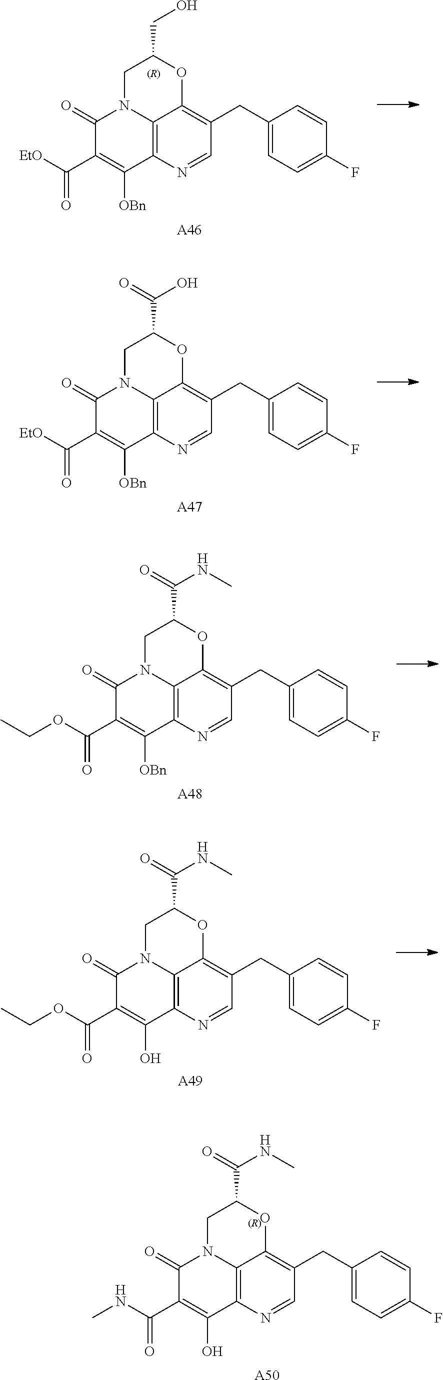

### Step 1

In a three-necked 1 L round bottom flask, (R)-(−)-2,2-Dimethyl-1,3-dioxolane-4-methanol A36 (9.85 g, 74.5 mmol, 1 equiv) was dissolved in anhydrous dichloromethane (275 mL) and cooled down to −40° C. in a cooling bath. To this solution was added 2,6-lutidine (9.5 mL, 82 mmol, 1.1 equiv) and, from an addition funnel, dropwise trifluoromethane sulfonic anhydride (22 g, 78.3 mmol, 1.05 equiv) over 10 min, monitoring the internal temperature with a probe. The reaction mixture was stirred 1 hour at −40° C. and then diluted with dichloromethane and washed with citric acid/sodium citrate buffer solution (pH=4). (To prepare this solution, 1 part solid citric acid and 1 part sodium citrate was dissolved in water and pH adjusted to 4-5). The aqueous layer was washed with dichloromethane and organic layers were combined and washed with brine and dried over sodium sulfate. The solvent was removed on rotovap and the colorless oil was dried on pump. Yield=20 g of compound A37 (crude).

### Step 2

4-Bromo-3-tert-butoxycarbonylamino-5-(4-fluoro-benzyl)-pyridine-2-carboxylic acid isopropyl ester A5 (15 g, 32 mmol) was dissolved in anhydrous DMF (70 mL) and the reaction flask was cooled down to −10° C. in a cooling bath. From an addition funnel was dropwise added 1M solution of sodium bis(trimethyl silyl) amide (42 mL, 42 mmol) over 5 minutes. A solution of the triflate compound A37 (13 g, 48 mmol) in DMF (40 mL) was then slowly added to the reaction mixture, keeping the internal temperature around −10° C. The mixture was stirred at −10° C. for 1 hour, and to the reaction mixture was then added excess ethyl acetate and organic layer was washed with citrate buffer solution at pH 4, and with brine. The aqueous layers were washed with ethyl acetate and organic layers were combined and washed with brine and dried over sodium sulfate. Volatiles were removed on rotovap and the desired product (brown oil) was dried on pump. The desired product A38 (16.5 g) was used as-is in the next step. MS m/z: 583.6 (M+1).

### Step 3

Compound A38 (16.5 g) in a round bottom flask which was heated up neat to 180° C. in a heating block. Throughout the reaction the system was negative pressurized with a vacuum pump. The reaction was monitored the HPLC and LCMS. After 3 hours the reaction was complete. The crude product mixture was then chromatographed on normal phase using ethylacetate/hexanes (Rf: 0.57 in 30% EtOAc/Hexanes) to obtain desired product A39 (9.5 g, 70%). MS m/z: 481.98 (M+1).

### Step 4

Compound A39 (9.5 g, 20 mmol, 1 equiv) was dissolved in 1,2-dichloroethane (200 mL) and to it was added ethyl malonyl chloride (2.77 mL, 22 mmol) at room temperature, followed by dropwise addition of a solution of 2,6-lutidine (2.55 mL, 22 mmol) in 1,2-dichloroethane over 10 min. The mixture was stirred about 1 hour at room temperature. LCMS confirmed the completion of the reaction. The reaction content was diluted with dichloromethane and washed with the citric acid buffer solution at pH 4, and with brine. The organic layers were combined and dried over sodium sulfate and concentrated down on rotovap and further dried on pump. The crude residue was then purified on a normal phase column (from 30% EtOAc/Hexanes to 65% EtOAc/Hexane in 1 hour). Purified Yield=10 g of compound A40 (85%). MS m/z: 596.26 (M+1).

### Step 5

Compound A40 (10 g, 16.8 mmol) was taken up in ethanol (160 mL) and to it was added 21% sodium ethoxide in ethanol (8 mL, 20.2 mmol). The mixture was stirred at room temperature for 1 hour. LCMS indicated a complete reaction. The ethanol was removed on rotovap and the residue was taken up in ethyl acetate and washed with citric acid solution buffered with sodium citrate at pH 4, twice. The organic layer was then washed with brine. The aqueous layers were combined and extracted with more ethyl acetate and the organic layers were combined and dried over sodium sulfate, and concentrated down on rotovap. The resulting residue was then further dried on pump. Yield=8 g of compound A41 (crude). The desired product was not attempted to purify and used as-is in the next step. MS m/z: 536.68 (M+1).

### Step 6

Compound A41 (7.5 g, 12 mmol) was dissolved in anhydrous dichloromethane and silver(I) oxide (5.57 g, 24 mmol) was added. The mixture was stirred for 20 minutes at room temperature. A solution of benzyl bromide (1.57 mL, 13.2 mmol,) in dichloromethane was dropwise added over 5 min and the reaction mixture was stirred overnight at room temperature. In the beginning of the reaction, some of the benzyl group will react with the 2-C carbonyl oxygen to give the unwanted regioisomer in addition to the 4C oxygen isomer, which is the desired isomer. Overnight reaction at room temperature will equilibrate the reaction towards the thermodynamic desired regioisomer as almost at a hundred percent rate. After overnight reaction the LCMS showed a complete conversion with single isomer. The wrong isomer was less than 2%. The crude mixture was then passed through a Celite plug and the filtrate was concentrated down on rotovap and taken up in ethyl acetate. The organic layer was then washed with brine a couple of times and dried over sodium sulfate. The volatiles were then removed and the residue was purified on normal phase column. (Rf=0.22 in 30% EtOAc/Hexanes). Purified Yield=7.8 g of compound A42 (90%). MS m/z: 626.73 (M+1).

### Step 7

Compound A42 (6.5 g, 10.4 mmol, 1 equiv) was treated with 60% acetic acid in water (260 mL), overnight at room temperature. After overnight reaction the HPLC data showed 96% completion. The reaction content was then slightly warmed to 45° C. in an oil bath and stirred for 3 more hours. HPLC then indicated a complete reaction. The crude reaction mixture was then reduced on rotovap as much as possible and then transferred into a seperatory funnel and washed (1V:1V) mixture of (brine:water) to remove most of excess acetic acid. Then the aqueous layer was checked by HPLC to make sure product did not escape there. The organic layer was then washed with sodium bicarbonate adjusting the pH to 8. Basic aqueous layer was extracted with ethyl acetate and the organic layers were combined and washed with brine and dried over sodium sulfate. Yield=5.4 g of compound A43 (90%).¹H NMR (300 MHz, CDCl₃) 8.34 (s, 1H), 7.45 (m, 2H), 7.36 (m, 3H), 7.12 (m, 2H), 7.0 (m, 2H), 5.59 (dd, Ja=7.6 Hz, Jb=10.8 Hz, 2H), 4.5 (m, 2H), 4.3 (m, 2H), 3.70 (m, 2H), 1.28 (t, J=6.8 Hz, 3H); MS m/z: 586.84 (M+1).

### Step 8

Compound A43 (5.4 g, 9.2 mmol) was dissolved in anhydrous dichloromethane (92 mL). To this solution was added triisopropyl silyl chloride (3.9 mL, 18.4 mmol, 2 equiv) and imidazole (1.25 g, 18.4 mmol), followed by DMAP (112 mg, 0.92 mmol). The mixture was stirred overnight at room temperature. After overnight reaction the HPLC showed the reaction went to completion. The volatiles were removed on rotovap and the residue was taken up in ethyl acetate and washed with citric acid solution buffered with sodium citrate at pH 4, once, and the aqueous layer was extracted with ethyl acetate. The combined organic layers were washed with brine and dried over sodium sulfate. The volatiles were removed and the residue dried on pump. The crude material was then purified on normal phase column with ethyl acetate/hexanes. (30% EtOAc/Hexanes, Rf=0.46). Purified Yield=4.4 g of compound A44 (65%). ¹H NMR ((400 MHz, CDCl₃) 8.32 (s, 1H), 7.46 (m, 2H), 7.36 (m, 3H), 7.15 (m, 2H), 7.02 (m, 2H), 5.58 (s, 2H), 4.84 (m, 2H), 4.35 (q, J=7.8 Hz, 2H), 4.26 (s, 2H), 4.13 (m, 1H), 3.74 (m, 2H), 1.57 (m, 3H), 1.30 (t, J=7.2 Hz, 3H), 1.05 (s, 18H); MS m/z: 741.21 (M+1).

### Step 9

Compound A44 (4 g, 5.4 mmol) was dissolved in anhydrous N,N-dimethylformamide (100 mL) and cooled down to −40° C. in a cooling bath. 1M Solution of sodium bis(trimethyl silyl)amide (5.9 mL, 5.9 mmol) was then dropwise added. The reaction was maintained at −40° C. for about 15 min and confirmed by LCMS to be complete. In addition to the desired product peak, LCMS also showed a smaller peak with the same molecular weight as the desired product. This byproduct peak was believed to be the corresponding 7-member cyclized byproduct. This byproduct was easily separated from the desired product by normal phase column chromatography. Yield=350 mg (10%). The crude mixture was diluted with ethyl acetate and washed with citric acid solution buffered with sodium citrate at pH 4, once, and the aqueous layer was extracted with ethyl acetate. The combined organic layers were washed with brine and dried over sodium sulfate. The crude product was then purified on normal phase column (30% EtOAc/Hexanes, Rf=0.4). Purified Yield=2.16 g of compound A45 (61%). ¹H NMR (400 MHz, CDCl₃) 8.30 (s, 1H), 7.44 (m, 2H), 7.32 (m, 3H), 7.17 (m, 2H), 6.95 (m, 2H), 5.66 (s, 2H), 4.7 (m, 1H), 4.32 (q, J=7.2 Hz, 2H), 4.21 (m, 1H), 4.02 (m, 4H), 3.75 (m, 2H), 1.6 (m, 7H), 1.27 (t, J=6.8 Hz, 3H), 1.05 (m, 18H); MS m/z: 661.35 (M+1).

### Step 10

Compound A45 (2 g, 3 mmol) was dissolved in THF/DI-water (1v:1v, 12 mL, 12 mL) and to it was added trifluoroacetic acid (4 mL). The mixture was stirred overnight at room temperature. LCMS showed a complete reaction. HPLC showed spot-to-spot conversion. Excess TFA and THF were reduced on rotovap as much as possible and the remaining slurry was extracted into ethyl acetate and the aqueous layers was extracted with ethylacetate until no desired peak was detected on HPLC of aqueous layer. The organic layers were combined and washed with saturated solution of sodium bicarbonate twice and with brine one time. The organic layer was then dried over sodium sulfate and further dried on pump. Yield=1.3 g of compound A46 (86%). ¹H NMR (300 MHz, CDCl₃) 8.35 (s, 1H), 7.47 (m, 2H), 7.36 (m, 3H), 7.20 (m, 2H), 7.01 (m, 2H), 5.71 (s, 2H), 4.7 (m, 1H), 4.35 (q, J=7.2 Hz, 2H), 4.28 (m, 1H), 4.07 (s, 2H), 3.95 (m, 2H), 3.75 (m, 1H), 1.30 (t, J=6.9 Hz, 3H); MS m/z: 505.19 (M+1).

### Step 11

To a solution of intermediate A46 (1.3 g, 2.6 mmol) in 1,2-dichloroethane (13 mL) and water (5 mL), was added iodobenzene diacetate (1.7 g, 5.2 mmol) and 2,2,6,6-tetramethylpiperidine-1-oxyl or TEMPO (81 mg, 0.52 mmol). The reaction was stirred at room temperature overnight. Upon completion, methanol (50 mL) was added to the reaction before being concentrated in vacuo. The crude residue was then trituration with EtOAc and Hexane. After filtration to give the white solid product A47 (1.1 g, 83%). ¹H NMR (400 MHz, DMSO-D6) δ 8.40 (s, 1H), 7.4-7.3 (m, 7H), 7.07 (m, 2H), 5.58 (dd, Ja=15.2 Hz, Jb=11.2 Hz, 2H), 5.34 (bs, 1H), 4.52 (m, 1H), 4.14 (q, J=6.8 Hz, 1H), 4.07 (m, 1H), 4.04 (s, 2H), 1.13 (t, J=6.8 Hz, 3H); MS m/z: 519.16 (M+1).

### Step 12

To a solution of an impure batch of carboxylic acid A47 (800 mg, 1.54 mmol) in DMF (15 mL) that had been stirred with HATU (1.1 g, 3 mmol) was treated with Methylamine (1.5 mL, 3 mmol, 2M THF solution) and N,N-diisopropylethylamine (1 mL, 6 mmol). The reaction mixture was stirred for 1 hour at room temperature, under nitrogen atmosphere. At which point, the reaction was diluted with ethyl acetate and quenched with saturated NH₄Cl. The organic layer was washed with brine, then dried (NaSO₄), filtered and concentrated. The residue was purified by chromatography on silica gel ((9/1-DCM/MeOH) to afford the desired product A48 (664 mg, 81%): 400 MHz ¹H NMR (CDCl₃) 8.39 (s, 1H), 7.44 (m, 2H), 7.34 (m, 3H), 7.20 (m, 2H), 7.03 (m, 2H), 5.67 (d, 2H), 4.74 (m, 2H), 4.32 (q, J=7.2 Hz, 2H), 4.09(s, 2H), 3.92(m, 1H), 2.70 (d, 3H), 1.28 (t, J=7.2 Hz, 3H); MS [M+H]=532.

### Step 13

To a solution of intermediate A48 (664 mg, 1.25 mmol) dissolved in EtOH:EtOAc (18 mL:9 mL) was added Palladium (10 wt % on carbon) [Pd/C] (100 mg). The reaction was run under hydrogen gas (using a balloon), purging several times with vacuum, at room temperature for 2 hours. At which point the reaction was degassed, and filtered to remove palladium (MeOH was used to completely solubilize the desired product), and concentrated in vacuo to afford the desired product A49 (523 mg, 95%): 400 MHz ¹H NMR (CD₃OD) 8.18(s, 1H), 7.21 (m, 2H), 6.94 (m, 2H), 4.91 (m, 1H), 4.50 (m, 1H),4.32 (q, J=7.2 Hz, 2H), 4.11(m, 2H), 3.92(m, 1H), 2.70 (s, 3H), 1.27 (t, J=7.2 Hz, 3H); MS [M+H]=442

### Step 14

To a solution of intermediate A49 (300 mg, 0.68 mmol) in DMF 6.8 mL) was added methylamine (2 m, 4 mmol). The reaction was heated in a microwave reactor at 125° C. for 20 minutes. Upon completion, after trituration to afford the desired product A50/Comparator Compound (218 mg, 75%; melting point 300° C.): 400 MHz ¹H NMR (DMSO) 10.05(m, 1H), 8.29(s, 1H), 8.11(m, 1H), 7.31 (m, 2H), 7.08 (m, 2H), 5.02 (m, 1H), 4.42 (m, 1H), 4.19-4.05 (m, 1H), 3.98 (m, 2H), 2.83 (d, 3H), 2.62 (d, 3H); MS [M+H]=427; Chiral HPLC: Chiralcel OD-H, MeOH/EtOH=50/50, Retention time: 26.7 min.

### EXAMPLE 4

### Antiviral Assays in MT2 and MT4 Cells

For the antiviral assay utilizing MT-2 cells, 50 μL of 2× test concentration of 5-fold serially diluted compound in culture medium with 10% FBS was added to each well of a 96-well plate (9 concentrations) in triplicate. MT-2 cells were infected with HIV-IIIb at a multiplicity of infection (m.o.i) of 0.01 for 3 hours.

Fifty microliters of infected cell suspension in culture medium with 10% FBS (˜1.5×10⁴ cells) was then added to each well containing 50 μL of diluted compound. The plates were then incubated at 37° C. for 5 days. For the antiviral assay utilizing MT-4 cells, 20 μL of 2× test concentration of 5-fold serially diluted compound in culture medium with 10% FBS was added to each well of a 384-well plate (7 concentrations) in triplicate. MT-4 cells were next mixed with HIV-IIIb at an m.o.i. of 0.1 and 20 μL of virus/cell mixture (˜2000 cells) was immediately added to each well containing 20 μL of diluted compound. The plates were then incubated at 37° C. for 5 days. After 5 days of incubation, 100 μL of CellTiter-Glo™ Reagent (catalog #G7571, Promega Biosciences, Inc., Madison, Wis.) was added to each well containing MT-2 cells and 40 μl to each well containing MT-4 cells. Cell lysis was carried out by incubating at room temperature for 10 min and then chemiluminescence was read.

EC₅₀ values are shown in Table 1.

TABLE 1
Potency of Compound (Ia)

| | | | |
|---|---|---|---|
| EC₅₀ | 3.4 nanomolar | CC₅₀ (MT-2) | 5.3 micromolar |
| | | CC₅₀ (MT-4) | 42 micromolar |

As reported in WO 2010/011959, Comparator Compound provides an EC₅₀ in MT-2 cells of 6.8 nM. The compounds, thus, are both potent anti-virals.

### EXAMPLE 5

### Melting Point by DSC

Using an appropriately calibrated Differential Scanning calorimeter, TA Instruments Q1000, 1-2 mg of the test compound was weighed in an aluminum pan, which was closed with a pin-holed cover. The pan was placed on the sample holder, and an empty aluminum pan closed with a pin-holed cover was placed on the reference pan holder. The temperature was ramped up from 30° C. to 300° C. at a rate of 10° C. per minute. Melting temperature was determined from the peak of the observed endotherm.

TABLE 2
Melting Point

| | |
|---|---|
| Compound (Ia) | 177° C. |
| Comparator Compound | 300° C. |

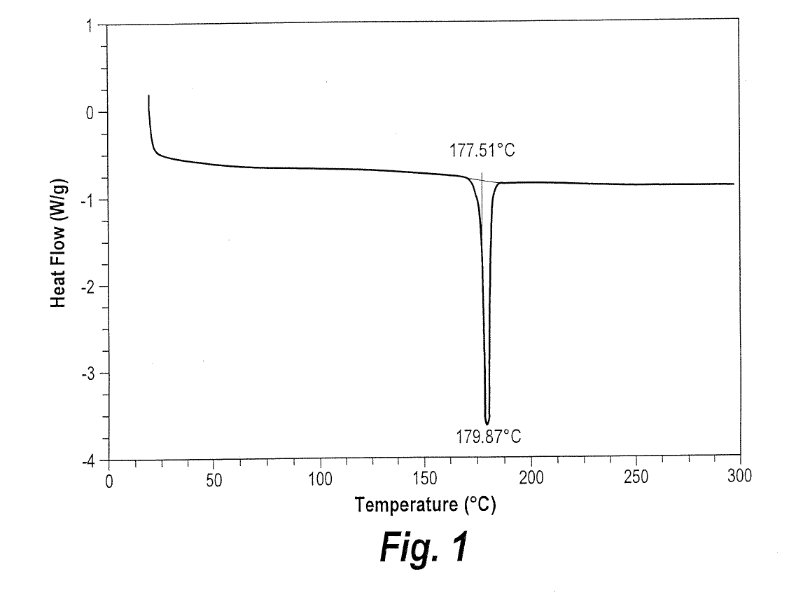

As demonstrated, Compound (Ia) has a significantly lower melting point than Comparator Compound. Additionally, as shown in FIG. 1, a DSC thermogram of Compound (Ia) demonstrates a well-defined, sharp endotherm.

### EXAMPLE 6

### Solubility

Excess solid material was added to aqueous buffers at pH 2 and pH 7 in glass vials to form suspensions. The suspension was stirred on a stirrer plate with a magnetic stirrer bar at room temperature for periods of 24 to 48 hours. The suspension was filtered through a 0.22 micron PVDF syringe filters. The clear filtrate was diluted in acetonitrile and analyzed by HPLC-UV against an external standard for the compound concentration.

TABLE 3
Aqueous solubility

| | |
|---|---|
| Compound (Ia) pH 7.3 | 31.6 μg/mL |
| Compound (Ia) pH 2.2 | 29.4 μg/mL |
| Comparator Compound pH 7.3 | 1 μg/mL |
| Comparator Compound pH 2.2 | 1 μg/mL |

As demonstrated, Compound (Ia) exhibits improved solubility over Comparator Compound.

### EXAMPLE 7

Oral Bioavailability—Determination of Oral Bioavailability of Integrase Inhibitors from Crystalline Suspension in Dog

Each of Compound (Ia) and Comparator Compound were synthesized in the Medicinal Chemistry Department at Gilead Sciences, Foster City, Calif.

### Study Details

The in-life phase of these studies was carried out at SRI, Menlo Park, Calif. Both Compound (Ia) and Comparator Compound were formulated in 0.5% HPMC E4M, 0.2% Tween 20, 0.9% benzyl alcohol, and 98.4% water. Both were at 2.00 mg/mL for oral dosing.

Each dosing group consisted of 3 male, beagle dogs. At dosing, the animals weighed an average of 10.9 kgs for Compound (Ia) and 10.5 kgs for Comparator Compound. The animals were fasted overnight prior to dose administration and up to 4 hr after dosing.

The dogs were pre-treated with pentagastrin at 30 minutes prior to dosing by IM injection. The Compound (Ia) suspension was administered by oral gavage at 1.5 mL/kg to deliver a total dose of 3 mg/kg, while the Comparator Compound suspension was administered by oral gavage at 2 mL/kg for a dose of 4 mg/kg.

Serial venous blood samples (approximately 0.8-1.0 mL each) were taken at specified time points (range t=0 to t=24 hr) after dosing from each animal. The blood samples were collected into Vacutainer™ tubes containing EDTA-K3 as the anti-coagulant and were immediately placed on wet ice pending centrifugation for plasma.

Dosing solutions were analyzed using un-used portions of the dosing solutions, and aliquots of approximately 1 mL of each dosing solution were saved. The concentration of compound in the dosing solution was measured with an HPLC-UV method. Comparator Compound (nominal dose of 4 mg/kg) was dosed at 9387 nmoles/kg, while Compound (Ia) (nominal dose of 3 mg/kg) was dosed at 6563-6566 nmoles/kg.

### Determination of Concentration of Test Compound in Plasma

An LC/MS/MS method was used to measure the concentration of test compounds in plasma. Non-compartmental pharmacokinetic analysis was performed on the plasma concentration-time data.

The bioanalytical method consisted of treating 100 μL of each plasma sample with 200 μL of acetonitrile (ACN) and 200 μL acetonitrile (ACN) containing internal standard. After protein precipitation and centrifugation, 100 μL of the supernatant was transferred to a clean 96-well plate and mixed with 100 μL of water. An aliquot of 10 μL of the above solution was injected onto an API-4000 triple quadrupole LC/MS/MS system. A Hypersil Gold (#25005-053030, Thermo Electron Corporation) column was used. Mobile phase A contained water with 0.1% formic acid and 1% IPA. Mobile phase B contained acetonitrile with 0.1% formic acid and 1% IPA.

### Results

TABLE 4
PK Parameters of Comparator Compound after oral dose of
Comparator Compound at 4 mg/kg in suspension in dogs
(mean ± SD, n = 3)

| PO | Comparator Compound | | | | |
|---|---|---|---|---|---|
| Plasma | Dog 3 | Dog 6 | Dog 7 | Mean | SD |
| AUCₗₐₛₜ (nM · hr) | 385 | 89.8 | 55.2 | 177 | 181 |
| AUC_{inf} (nM · hr) | 427 | NC | NC | 427 | NC |
| Cₘₐₓ (nM) | 58.5 | 16.3 | 11.3 | 28.7 | 25.9 |
| Tₘₐₓ (hr) | 4.00 | 2.00 | 2.00 | 2.67 | 1.15 |
| t_{1/2} (hr) | 2.97 | NC | NC | NC | NC |
| CL (L/hr/kg) | 0.13 | 0.14 | 0.19 | 0.15 | 0.03 |
| F (%) | 0.5% | 0.1% | 0.1% | 0.2% | 0.2% |

TABLE 5
PK Parameters of Compound (Ia) after oral dose of Compound (Ia)
at 3 mg/kg in suspension in dogs (mean ± SD, n = 3)

| PO | Compound (Ia) | | | | |
|---|---|---|---|---|---|
| Plasma | Dog 12 | Dog 16 | Dog 17 | Mean | SD |
| AUCₗₐₛₜ | 19082 | 14199 | 21762 | 18348 | 3835 |
| (nM · hr) | | | | | |
| AUC_{inf} | 19335 | 14413 | 21913 | 18554 | 3810 |
| (nM · hr) | | | | | |
| Cₘₐₓ (nM) | 3160 | 2500 | 4240 | 3300 | 878 |
| Tₘₐₓ (hr) | 1.00 | 1.00 | 2.00 | 1.33 | 0.58 |
| t_{1/2} (hr) | 4.16 | 4.88 | 3.64 | 4.22 | 0.62 |
| CL (L/hr/kg) | 0.17 | 0.15 | 0.17 | 0.16 | 0.01 |
| F (%) | 47.0% | 35.0% | 53.6% | 45.2% | 9.44% |

### Summary

After the oral administration of Comparator Compound in 0.5% HPMC E4M, 0.20% Tween 20, 0.9% Benzyl Alcohol, and 98.4% water in pentagastrin pretreated dogs at 4 mg/kg, the Cₘₐₓ was 28.7±25.9 nM and the AUC₀₋ₜ was 177±181 nM·hr. The oral bioavailability was 0.2%±0.2%.

After the oral administration of Compound (Ia) at 3 mg/kg as a suspension consisting of 0.5% HPMC E4M, 0.2% Tween 20, 0.9% Benzyl Alcohol, 98.4% water in pentagastrin pretreated dogs, Compound (Ia) reached a Cₘₐₓ of 3300±878 nM at approximately 1.33 hr, and the oral bioavailability was 45%±9%.

As demonstrated, Compound (Ia) provides a substantial improvement in oral bioavailability as compared to Comparator Compound. Thus, although each compound demonstrates a preferred level of potency, the lower melting point and increased solubility of Compound (Ia) further provides improved oral bioavailability.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

The invention has been described with reference to various specific and preferred embodiments and techniques. However, it should be understood that many variations and modifications may be made while remaining within the spirit and scope of the invention.

What is claimed:

1. A compound of Formula (I): 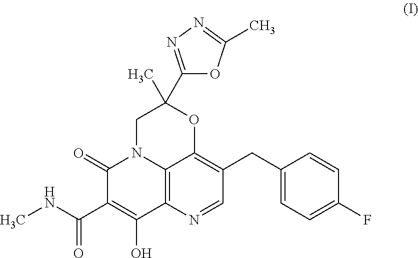
or a pharmaceutically acceptable salt thereof.

2. A compound of Formula (Ia): 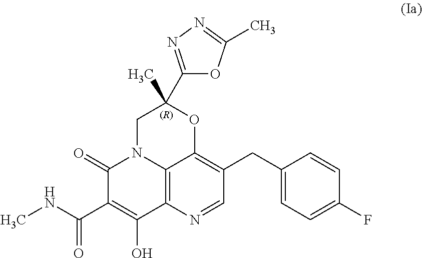
or a pharmaceutically acceptable salt thereof.

3. A pharmaceutical composition comprising the compound of claim 1 or 2 and one or more pharmaceutically acceptable excipients.

4. The pharmaceutical composition of claim 3, further comprising one or more additional therapeutic agents.

5. The pharmaceutical composition of claim 4, wherein the additional therapeutic agent is selected from the group consisting of HIV protease inhibiting compounds, HIV non-nucleoside inhibitors of reverse transcriptase, HIV nucleoside inhibitors of reverse transcriptase, HIV nucleotide inhibitors of reverse transcriptase, HIV integrase inhibitors, gp41 inhibitors, CXCR4 inhibitors, entry inhibitors, gp120 inhibitors, G6PD and NADH-oxidase inhibitors, CCR5 inhibitors, interferons, ribavirin analogs, NS5a inhibitors, NS5b inhibitors, NS3 protease inhibitors, alpha-glucosidase 1 inhibitors, hepatoprotectants, non-nucleoside inhibitors of HIV, capsid inhibitors, inhibitors of the binding of host factor LEDGF to HIV integrase, and pharmacokinetic enhancers.

6. A method for delaying onset or progression of disorders associated with HIV infection in a human, comprising administering a therapeutically effective amount of a compound of claim 1 or 2.

7. A method for the treatment of HIV infection in a human, comprising administering a therapeutically effective amount of a compound of claim 1 or 2.

8. A compound of claim 1 or 2 for use in medical therapy.

9. A compound of claim 1 or 2 for use in treating or preventing HIV, or an HIV-associated disorder.

10. The composition of claim 3 or 5, wherein the composition is provided in a once-daily administration.

11. The composition of claim 3 or 5, wherein the composition is provided in a twice-daily administration.

12. The composition of claim 3 or 5 or 11-12, wherein the dose ranges from:
a. about 1 to about 6 mg/kg body weight per day for monotherapy; and
b. about 1 to about 20 mg/kg body weight per day for combination therapy.
